(19) United States
(12) Patent Application Publication
(10) Pub. No.: US 2016/0331315 A1
(43) Pub. Date: Nov. 17, 2016

(54) Systems and Methods for Wearable Health Monitoring

(71) Applicants: Abraham Carter, Palo Alto, CA (US); David Scott, North Salt Lake City, UT (US); Ehsan Azarnasab, Palo Alto, CA (US)

(72) Inventors: Abraham Carter, Palo Alto, CA (US); David Scott, North Salt Lake City, UT (US); Ehsan Azarnasab, Palo Alto, CA (US)

(21) Appl. No.: 15/061,528

(22) Filed: Mar. 4, 2016

Related U.S. Application Data
(60) Provisional application No. 62/161,653, filed on May 14, 2015.

(51) Int. Cl. A61B 5/00; A61B 5/0205; A61B 5/11

(52) U.S. Cl. CPC A61B 5/6801; A61B 5/1118; A61B 5/0022; A61B 5/4806; A61B 5/742; A61B 5/746; A61B 5/7246; A61B 5/7282; A61B 5/0205; A61B 5/7275; A61B 2562/0219; A61B 5/02433

(57) ABSTRACT

Aspects of the present invention relate to recording physiological data sequences while a user participates in various activities. These activities may comprise awake activities, periods of sleep and transitions between sleep and activity. Record sequences are recorded and maintained for a period of time including many cycles of activity. The physiological activity data sequences may then be analyzed to identify trends that lead up to specific events that have occurred. The physiological data may also be compared to known trends to alert a user to trends that may lead to adverse events. Some embodiments comprise physiological sequence records that document multiple sleep and activity cycles over an extended period of time.

### CROSS-REFERENCE TO RELATED APPLICATION

This application claims the benefit of priority from U.S. Provisional Patent Application No. 62/161,653 which was filed on May 14, 2015.

### BACKGROUND OF THE INVENTION

1. Field of the Invention

The present invention relates to a method and apparatus for non-invasively tracking physiological characteristics over many cycles of sleep and activity.

2. Background and Related Art

Currently, methods and apparatus exist for monitoring an individual's physiological characteristics. However, these methods and apparatus are typically employed in a doctor's office or laboratory environment where they are applied to an individual for a brief period of time and involve the use of bulky, dedicated equipment. These methods can also typically require a user to perform a specific activity on a specific piece of equipment for a specific period of time thereby leaving a user with no alternatives. These current methods pose significant challenges to users with physical challenges.

Current methods and apparatus for testing during sleep can also require a patient to visit and unfamiliar laboratory environment for testing. The unfamiliar environment can introduce anomalies into the testing process that make test results more difficult to interpret.

### BRIEF SUMMARY OF THE INVENTION

A method and apparatus are provided for recording physiological data sequences while a user participates in periods of sleep and various activities and transitions between sleep and activity. Record sequences are recorded and maintained for a period of time including many cycles of activity. The physiological activity data sequences may then be analyzed to identify trends that lead up to specific events that have occurred. The physiological data may also be compared to known trends to alert a user to trends that may lead to adverse events. Some embodiments comprise physiological sequence records that document multiple sleep and activity cycles over an extended period of time.

### BRIEF DESCRIPTION OF THE SEVERAL VIEWS OF THE DRAWINGS

The objects and features of the present invention will become more fully apparent from the following description and appended claims, taken in conjunction with the accompanying drawings. Understanding that these drawings depict only typical embodiments of the invention and are, therefore, not to be considered limiting of its scope, the invention will be described and explained with additional specificity and detail through the use of the accompanying drawings in which:

FIG. 1 shows an exemplary general-purpose computer system;

FIG. 2 shows a representative networked system configuration related to embodiments of the present invention;

FIG. 3 shows an exemplary wearable component of the present invention;

FIG. 4 shows the communication connections of an embodiment of the present invention;

FIG. 5 shows exemplary data communicated between components of the present invention;

FIG. 6 shows an exemplary process of the present invention wherein data is recorded for a fixed period of time before analysis occurs;

FIG. 7 shows an exemplary process of the present invention wherein data is recorded until a predetermined event occurs;

FIG. 8 shows an exemplary sequence record of an embodiment of the present invention.

### DETAILED DESCRIPTION OF THE INVENTION

A description of embodiments of the present invention will now be given with reference to the Figures. It is expected that the present invention may take many other forms and shapes, hence the following disclosure is intended to be illustrative and not limiting, and the scope of the invention should be determined by reference to the appended claims.

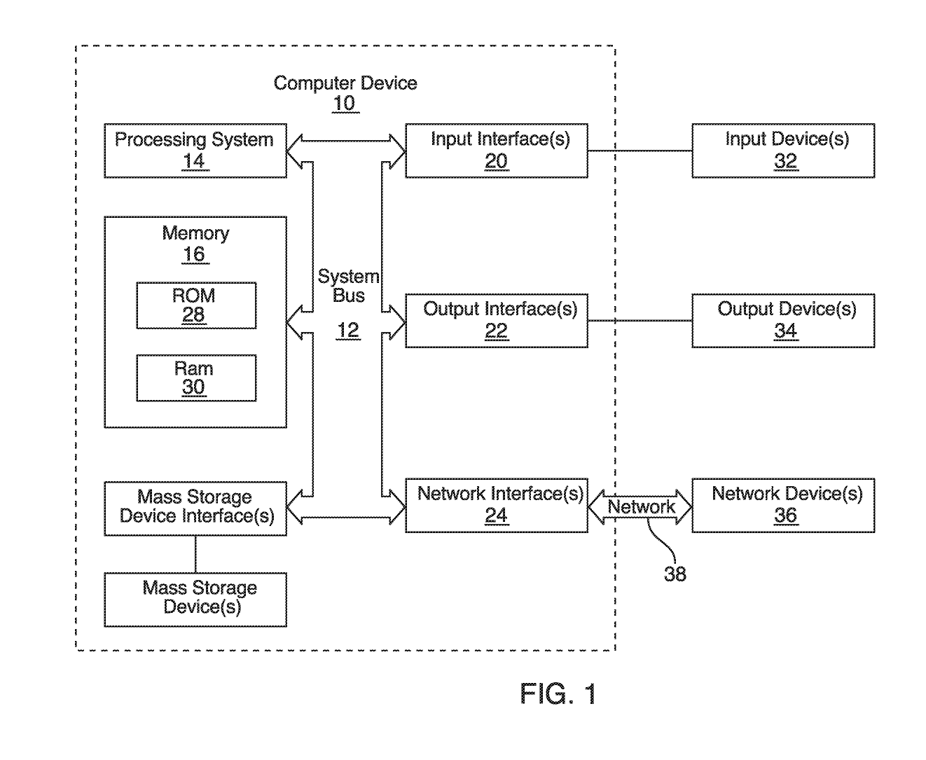

FIG. 1 and the corresponding discussion are intended to provide a general description of a suitable operating environment in which embodiments of the invention may be implemented. One skilled in the art will appreciate that embodiments of the invention may be practiced by one or more computing devices and in a variety of system configurations, including in a networked configuration. However, while the methods and processes of the present invention have proven to be useful in association with a system comprising a general purpose computer, embodiments of the present invention include utilization of the methods and processes in a variety of environments, including embedded systems with general purpose processing units, digital/media signal processors (DSP/MSP), application specific integrated circuits (ASIC), stand alone electronic devices, and other such electronic environments.

Embodiments of the present invention embrace one or more computer-readable media, wherein each medium may be configured to include or includes thereon data or computer executable instructions for manipulating data. The computer executable instructions include data structures, objects, programs, routines, or other program modules that may be accessed by a processing system, such as one associated with a general-purpose computer capable of performing various different functions or one associated with a special-purpose computer capable of performing a limited number of functions. Computer executable instructions cause the processing system to perform a particular function or group of functions and are examples of program code means for implementing steps for methods disclosed herein. Furthermore, a particular sequence of the executable instructions provides an example of corresponding acts that may be used to implement such steps. Examples of computer-readable media include random-access memory (“RAM”), read-only memory (“ROM”), programmable read-only memory (“PROM”), erasable programmable read-only memory (“EPROM”), electrically erasable programmable read-only memory (“EEPROM”), compact disk read-only memory (“CD-ROM”), or any other device or component that is capable of providing data or executable instructions that may be accessed by a processing system. While embodiments of the invention embrace the use of all types of computer-readable media, certain embodiments as recited in the claims may be limited to the use of tangible, non-transitory computer-readable media, and the phrases “tangible computer-readable medium” and “non-transitory computer-readable medium” (or plural variations) used herein are intended to exclude transitory propagating signals per se.

With reference to FIG. 1, a representative system for implementing embodiments of the invention includes computer device 10, which may be a general-purpose or special-purpose computer or any of a variety of consumer electronic devices. For example, computer device 10 may be a personal computer, a notebook or laptop computer, a netbook, a personal digital assistant (“PDA”) or other hand-held device, a smart phone, a tablet computer, a workstation, a minicomputer, a mainframe, a supercomputer, a multi-processor system, a network computer, a processor-based consumer electronic device, a computer device integrated into another device or vehicle, or the like.

Computer device 10 includes system bus 12, which may be configured to connect various components thereof and enables data to be exchanged between two or more components. System bus 12 may include one of a variety of bus structures including a memory bus or memory controller, a peripheral bus, or a local bus that uses any of a variety of bus architectures. Typical components connected by system bus 12 include processing system 14 and memory 16. Other components may include one or more mass storage device interfaces 18, input interfaces 20, output interfaces 22, and/or network interfaces 24, each of which will be discussed below.

Processing system 14 includes one or more processors, such as a central processor and optionally one or more other processors designed to perform a particular function or task. It is typically processing system 14 that executes the instructions provided on computer-readable media, such as on memory 16, a magnetic hard disk, a removable magnetic disk, a magnetic cassette, an optical disk, or from a communication connection, which may also be viewed as a computer-readable medium.

Memory 16 includes one or more computer-readable media that may be configured to include or includes thereon data or instructions for manipulating data, and may be accessed by processing system 14 through system bus 12. Memory 16 may include, for example, ROM 28, used to permanently store information, and/or RAM 30, used to temporarily store information. ROM 28 may include a basic input/output system (“BIOS”) having one or more routines that are used to establish communication, such as during start-up of computer device 10. RAM 30 may include one or more program modules, such as one or more operating systems, application programs, and/or program data.

One or more mass storage device interfaces 18 may be used to connect one or more mass storage devices 26 to system bus 12. The mass storage devices 26 may be incorporated into or may be peripheral to computer device 10 and allow computer device 10 to retain large amounts of data. Optionally, one or more of the mass storage devices 26 may be removable from computer device 10. Examples of mass storage devices include hard disk drives, magnetic disk drives, tape drives and optical disk drives. A mass storage device 26 may read from and/or write to a magnetic hard disk, a removable magnetic disk, a magnetic cassette, an optical disk, or another computer-readable medium. Mass storage devices 26 and their corresponding computer-readable media provide nonvolatile storage of data and/or executable instructions that may include one or more program modules such as an operating system, one or more application programs, other program modules, or program data. Such executable instructions are examples of program code means for implementing steps for methods disclosed herein.

One or more input interfaces 20 may be employed to enable a user to enter data and/or instructions to computer device 10 through one or more corresponding input devices 32. Examples of such input devices include a keyboard, touchpad, dedicated buttons, mouse, trackball, light pen, stylus, or other pointing device, a microphone, a joystick, a game pad, a satellite dish, a scanner, a camcorder, a digital camera, and the like. Similarly, examples of input interfaces 20 that may be used to connect the input devices 32 to the system bus 12 include a serial port, a parallel port, a game port, a universal serial bus (“USB”), an integrated circuit, a firewire (IEEE 1394), or another interface. For example, in some embodiments input interface 20 includes an application specific integrated circuit (ASIC) that is designed for a particular application. In a further embodiment, the ASIC is embedded and connects existing circuit building blocks.

One or more output interfaces 22 may be employed to connect one or more corresponding output devices 34 to system bus 12. Examples of output devices include a monitor or display screen, a speaker, a printer, a multi-functional peripheral, and the like. A particular output device 34 may be integrated with or peripheral to computer device 10. Examples of output interfaces include a video adapter, an audio adapter, a parallel port, and the like.

One or more network interfaces 24 enable computer device 10 to exchange information with one or more other local or remote computer devices, illustrated as computer devices 36, via a network 38 that may include hardwired and/or wireless links. Examples of network interfaces include a network adapter for connection to a local area network (“LAN”) or a modem, wireless link, or other adapter for connection to a wide area network (“WAN”), such as the Internet. The network interface 24 may be incorporated with or peripheral to computer device 10. In a networked system, accessible program modules or portions thereof may be stored in a remote memory storage device. Furthermore, in a networked system computer device 10 may participate in a distributed computing environment, where functions or tasks are performed by a plurality of networked computer devices.

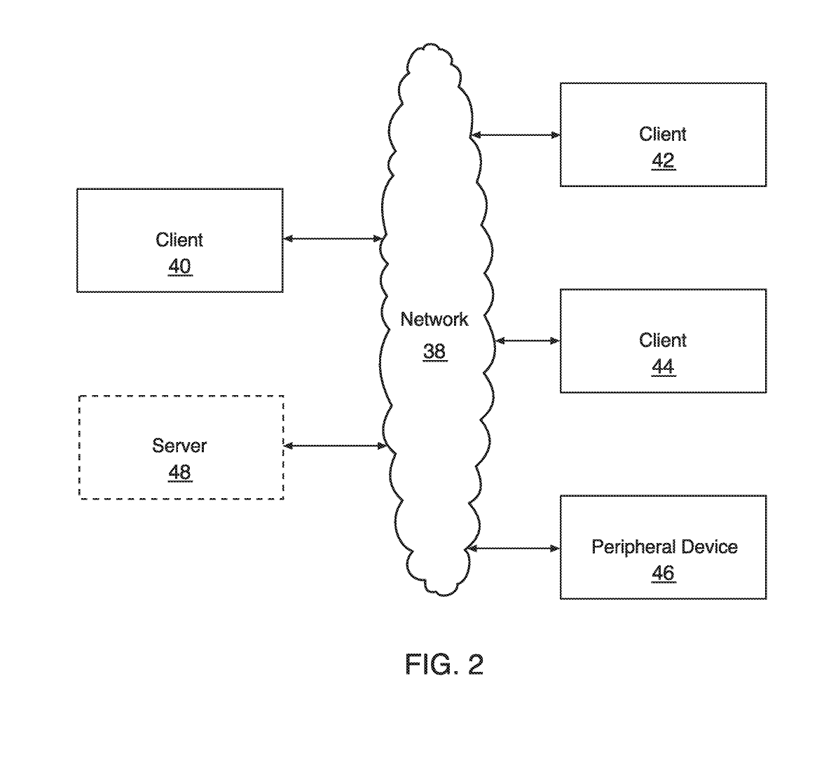

Thus, while those skilled in the art will appreciate that embodiments of the present invention may be practiced in a variety of different environments with many types of system configurations, FIG. 2 provides a representative networked system configuration that may be used in association with embodiments of the present invention. The representative system of FIG. 2 includes a computer device, illustrated as client 40, which is connected to one or more other computer devices (illustrated as client 42 and client 44) and one or more peripheral devices (illustrated as multifunctional peripheral (MFP) MFP 46) across network 38. While FIG. 2 illustrates an embodiment that includes a client 40, two additional clients, client 42 and client 44, one peripheral device, MFP 46, and optionally a server 48, connected to network 38, alternative embodiments include more or fewer clients, more than one peripheral device, no peripheral devices, no server 48, and/or more than one server 48 connected to network 38. Other embodiments of the present invention include local, networked, or peer-to-peer environments where one or more computer devices may be connected to one or more local or remote peripheral devices. Moreover, embodiments in accordance with the present invention also embrace a single electronic consumer device, wireless networked environments, and/or wide area networked environments, such as the Internet.

Similarly, embodiments of the invention embrace cloud-based architectures where one or more computer functions are performed by remote computer systems and devices at the request of a local computer device. Thus, returning to FIG. 2, the client 40 may be a computer device having a limited set of hardware and/or software resources. Because the client 40 is connected to the network 38, it may be able to access hardware and/or software resources provided across the network 38 by other computer devices and resources, such as client 42, client 44, server 48, or any other resources. The client 40 may access these resources through an access program, such as a web browser, and the results of any computer functions or resources may be delivered through the access program to the user of the client 40. In such configurations, the client 40 may be any type of computer device or electronic device discussed above or known to the world of cloud computing, including traditional desktop and laptop computers, smart phones and other smart devices, tablet computers, or any other device able to provide access to remote computing resources through an access program such as a browser.

To minimize the need to download and/or install programs on users' computing devices, embodiments of the invention utilize existing web browser technology. Many browser programs currently exist or are under development, and it would be impossible to name all such browser programs, but examples of such programs include Microsoft's Internet Explorer, Mozilla Firefox, Google Chrome, Apple Safari, Opera Software's Opera browser, as well as myriad browsers specifically configured for specific devices, such as Internet-connected smart phones and the like. The exact display of each browser can vary from browser to browser and most are moderately to highly configurable so as to vary the exact display,

Many currently-available browser programs permit the installation of additional features, such as through what are commonly known as “browser extensions.” Browser extensions are becoming more and more common in today's browser programs, and have become one of if not the standard for extending the functionality of the browser programs. For browsers that do not currently support browser extensions, other mechanisms and installed programs are often available to provide similar functionality.

Embodiments of the invention may utilize a browser extension or similar format to provide functions in accordance with embodiments of the invention. The use and installation of a browser extension is typically significantly less involved and less computer-intensive than the use and installation of a stand-alone program. In many instances, the installation of the browser extension occurs essentially without the computer's operating system being made aware of any additional installation. Instead, the browser program itself handles the browser extension and any demands made by the browser extension.

Embodiments of the present invention may comprise sensors and/or emitters for measuring physical and physiological characteristics of users and other parameters. Some embodiments may comprise accelerometers for measuring the proper acceleration of parts of a user's body or equipment used by the user. In some embodiments, multiple accelerometers may be aligned to measure acceleration along the axes of a 2- or 3-dimensional orthogonal grid, thereby allowing measurement of motion and acceleration in multiple dimensions. In some embodiments, accelerometers may be placed at multiple locations on a user's body to determine the relative motion of those body parts.

Acceleration data obtained from these sensors can be used to estimate or predict an activity being performed by the user. In some embodiments, an action measured by accelerometers positioned on a human body can be correlated with a record of known motions to determine what activity is being performed. Some embodiments may utilize methods and systems identified in U.S. patent application Ser. No. 20130282324, Matching System for Correlating Accelerometer Data to Known Movements, by Abraham Carter et al. is hereby incorporated herein by reference.

These accelerometers may be contained within a wearable device. A wearable device may comprise an article of clothing, a piece of jewelry, a hat, shoes, or a device that can be attached to something on the human body, such as a shoe clip, hair clip, wristband, etc. Exemplary wearable devices comprise, headbands, hats, necklaces, shirts, arm bands, wrist bands, vests, belts, pants, gloves, socks, shoes, watches and other devices.

Some embodiments of the present invention may comprise a heart rate monitor. A heart rate monitor may comprise sensors for measuring heart activity. In some embodiments, the electrical activity of the heart is sensed by sensors in the heart rate monitor to measure heart beats. This heart beat data may be measured at the sensor and sent wirelessly to a receiver on another device. Heart rate monitors of embodiments of the present invention may be contained within a wearable device similarly to the accelerometer sensors described above.

Some embodiments of the present invention may comprise a photoplethysmographic (PPG) sensor, which measures blood volume changes in microvascular tissue. A PPG sensor or pulse oximeter may comprise light emitters, such as light emitting diodes (LEDs) that may emit light in multiple frequencies (typically, red and infrared) and measure the difference in the intensity of light received on the other side of the vascular tissue. During a cardiac cycle the blood pressure increases and decreases with the pumping of the heart, these pressure changes expand and contract the arteries causing volumetric changes in the vascular tissue and corresponding changes in tissue volume and absorbed light. The difference in light transmitted through the tissue during a cardiac cycle determines the heart beat profile or PPG profile. Some wearable devices of the present invention may comprise a PPG sensor otherwise known as a pulse oximeter or photoplethysmograph.

The PPG signal may also be used to measure or estimate other physiological parameters. In some embodiments, respiration rate, respiration volume, intrapleural pressure, sinus arrhythmia and other parameters can be calculated from PPG measurements. In some embodiments, the depth of anesthesia and hypo- or hyper-volemic conditions can be measured based on the PPG signal.

Some embodiments of the present invention may comprise a blood glucose sensor for determining the blood glucose level of a user. This sensor may comprise a light-based sensor, similar to the PPG sensor, but measuring blood sugar level, using a light emitter and sensor. Some embodiments may comprise sensors using ultrasound, electromagnetic and thermal sensors to determine blood sugar levels.

Some embodiments of the present invention may comprise sensors that measure a galvanic skin response (GSR) or electrodermal activity. GSR sensors may measure a galvanic skin resistance as an electrical resistance between two electrodes on the surface of the skin and may measure a galvanic skin potential as a voltage between two electrodes on the surface of the skin without any externally applied current. A GSR value may comprise a combination of a skin resistance value and a skin potential value.

In some embodiments GSR values may be obtained at or between specific meridian points on the human body. These meridian points are locations on the surface of the skin that correspond to locations on specific energy or healing pathways.

Some embodiments of the present invention may comprise one or more sensors for measuring a hydration level of a user. These sensors may function similarly to the PPG sensor and/or the GSR sensor with circuitry or logic for correlating the basic signal to a hydration level to indicate the level of hydration of a user.

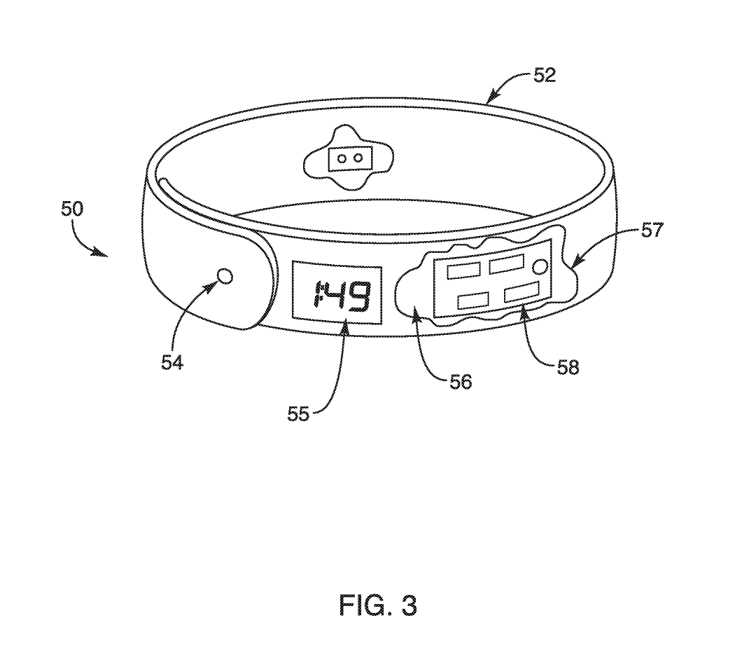

A wearable component of some embodiments of the present invention may be described with reference to FIG. 3. Wearable component 50 may comprise a wristband, anklet, finger ring, toe ring, belt, necklace, chest strap, arm band, garter, shirt, pants, underwear, bra, headband, shoe clip, wrap, strap, band, adhesive strip, bandage or other clothing or device worn or affixed on a part of the human body. Exemplary embodiments of wearable component 50 may be designed to be comfortable with a minimal form factor such that they do not impede motion, rest or other user activity. Some embodiments may be virtually unnoticeable and can be worn over multiple 24 hour periods with no discomfort or activity inhibition.

Some embodiments of wearable component 50 may comprise an adjustable closure 54 for fitting and securing the wearable component 50 to a part of the human body, such as a wrist or ankle. Wearable component 50 may further comprise circuitry 58, 60, which may comprise a microprocessor, memory, motion sensors, other sensors, emitters, antennas, power sources and other circuitry. In an exemplary embodiment, wearable component 50 may comprise motion sensors for detecting motion of wearable component 50 in one or more dimensions.

Some embodiments may also comprise a blood oximeter for detecting blood oxygen levels of a wearer. Some embodiments may comprise an emitter 60 and opposing sensor 58 for emitting a form of radiation, for example red and/or infrared light, and measuring one or more changes in that radiation as it passes through an appendage of the wearer. Some embodiments of wearable component 50 may comprise a heartbeat sensor and/or PPG sensor. Some embodiments of wearable component 50 may measure detailed heartbeat data for determining heartbeat profiles and volumetric blood flow data.

Some embodiments of wearable component 50 may comprise a GSR sensor, a hydration level sensor and/or a blood glucose level sensor as described above. Some embodiments of wearable component 50 may also comprise a display unit 55 for displaying information to a user, such as physiological parameters or alerts. Some embodiments of wearable component 50 may comprise an audio output device 57 for warnings, alerts, alarms, simulated voice communication or other audio communication.

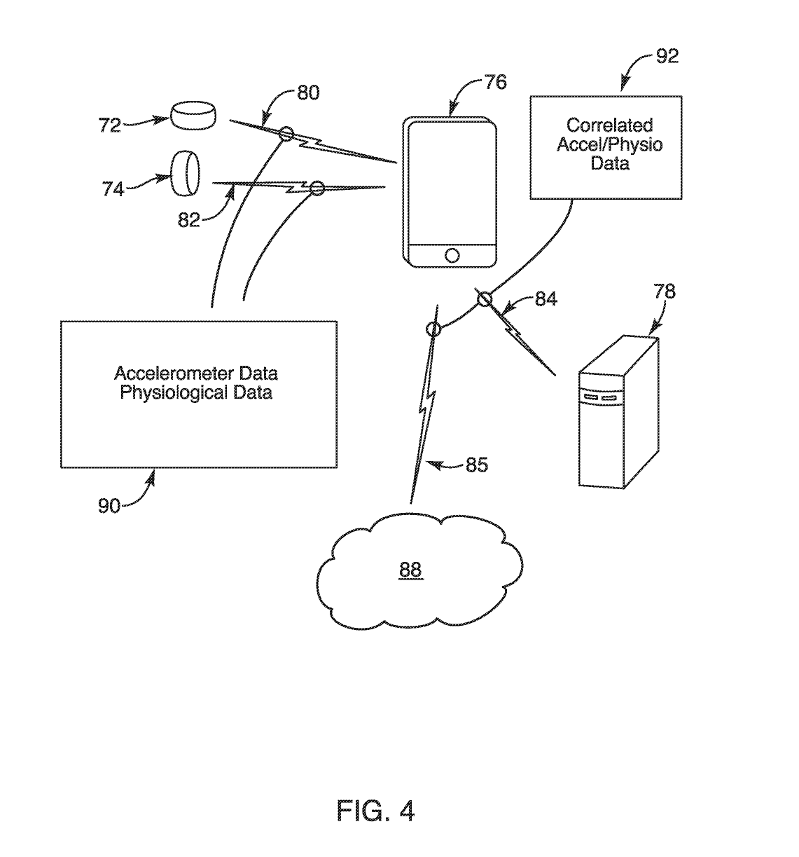

Some embodiments of the present invention may be described with reference to FIG. 4. These embodiments comprise one or more wearable components 72, 74 that may be worn by a user 70. These wearable components 72, 74 may comprise an article of clothing or some other form that is readily attachable to parts of the human body as discussed above. In the illustrated embodiment of FIG. 4, a first wearable component 72 is illustrated as a wristband and a second wearable component 74 is illustrated as an ankle band or shoe clip.

In some embodiments, a user 70 may also wear or carry a mobile computing device 76 such as a smart phone or similar lightweight, portable device. Some embodiments may further comprise one or more off-body computing devices 78.

Wearable components 72 and 74, mobile computing device 76 and off-body computing device 78 may comprise wireless transmitters, receivers or transceivers for one- or two-way communication between devices. In an exemplary embodiment, first wearable component 72 may establish a first wearable-to-mobile (W/M1) wireless connection 80 with a mobile computing device 76. A second wearable component 74 may also establish a second wearable-to-mobile (W/M2) wireless connection 82 with mobile computing device 76. First wearable component 72 and second wearable component 74 may also establish a wearable-to-wearable (W/W) wireless connection 86 for communication between wearable components 72, 74.

Some embodiments may further comprise a mobile-to-off-body computing device (M/O) wireless connection 84 for communication between a mobile computing device 76 and an off-body computing device 78.

These wireless communication connections 80, 82, 84, 86 may be established using known wireless communication protocols and methods, such as IEEE 802.11 (b), (g), (e), Bluetooth, ANT, wireless telephony (e.g., cell phone, satellite phone) and other methods. In an exemplary embodiment, connections 80, 82, 86 between on-body devices such as first and second wearable components 72, 74 and mobile computing device 76 may be established using a Bluetooth connection while communication between on-body devices 72, 74, 76 and off-body computing device 78, such as M/O connection 84 may be established using a cell phone data connection.

In some embodiments, wearable components 72, 74 may communicate directly with an off-body computing device 78 over a wireless connection (not shown). These embodiments may comprise an off-body computing device mounted to equipment such as a bicycle, elliptical exercise machine or other apparatus. Other embodiments may comprise an off-body computing device 78 similar to a desk-top computer, but which is within wireless communication range of a user during an activity, such as during sleep monitoring or other stationary activities.

Accelerometer-Identified Activity Data or General Activity Data Correlated with Physiological Data

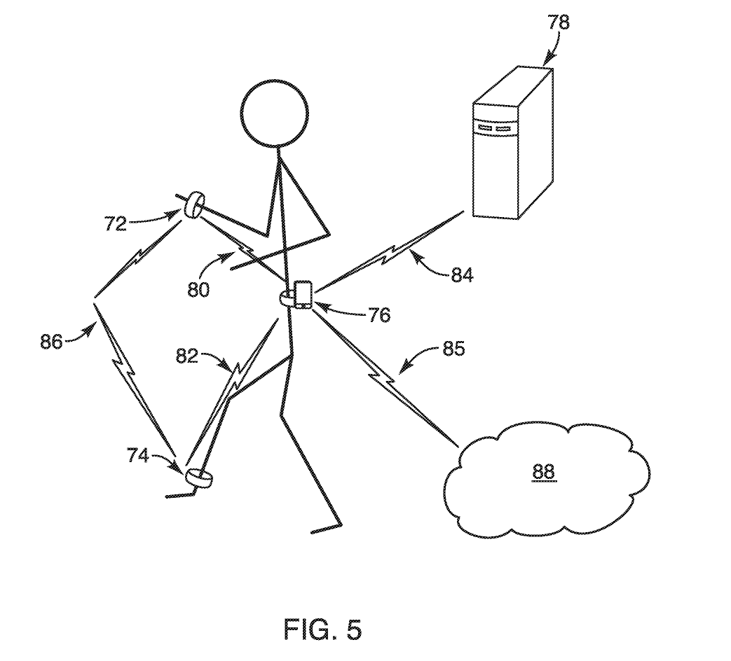

Some embodiments of the present invention may be described with reference to FIG. 5. In these embodiments, wearable components 72, 74 measure and transmit user motion and physiological characteristics using their various accelerometers and sensors as described above. This raw data may be transmitted 80, 82 directly to a mobile computing device 76 or another device as described above. In some embodiments, the raw data may be transmitted directly to mobile device 76 where the data may be recorded and processed. In these embodiments, the accelerometer data can be correlated with known movement data stored on the mobile device 76 to identify particular user activities as described in U.S. patent application Ser. No. 20130282324, incorporated hereinabove. In other embodiments, this processing can be performed at an off-body computing device 78 or a cloud-based system 88 over one or more wireless data connections 84, 85. If processing is performed on the mobile device 76, the correlated data 92 may be sent to an off-body computing device 78 or cloud system 88 for further processing and storage.

Furthermore, the correlated activity data or uncorrelated raw motion data may further be correlated with the user's physiological data to determine the user's overall physiological state during the identified activity or motion condition. For example, when an activity has been identified (such as running uphill at 3% grade, 6 mph, temp 72 deg, 1 hour into workout) the user's physiological state can be assessed based on past user data or general parameters and recorded for future comparison. After sufficient data collection for a particular user's activities, real time data can be compared to past performance data to determine if a user's physiological condition is normal, improving, abnormal, deteriorating, dangerous or another condition.

In some embodiments, a user's physiological state can be tracked and used as a performance guide indicating when a user's performance has improved or worsened during a specific activity under specific conditions. For example, if the user's physiological state has improved (lower heart rate, respiration rate) for the same activity under the same conditions, the user can be assured that their fitness level has increased. Likewise, a deterioration in physiological condition for a specific activity under the same conditions may indicate a serious health problem is occurring or imminent.

When motion data is not directly correlated with an identified activity, the motion data may still be assessed to determine a general state of activity. Raw motion data may be translated to a general indicator of activity level based on motion amplitude, frequency, energy, power, entropy, intensity or other parameters. In some embodiments, this general activity level can be correlated to a physiological condition as well. For example, an energy output level can be determined based on general user extremity motion and this energy output level can be correlated with a heart rate, blood glucose level, blood flow rate, respiration rate or another parameter or combination of parameters.

Physiological Data Correlated with Trends—Track Deviation Alert

Using embodiments of the present invention, a user can track physiological characteristics 24 hours a day for an indefinite period of time. This convenience is due to the comfort, non-intrusiveness and portability of the wearable component 50 and its wireless connections. If one or more wearable components are worn and activated, a user can track sleep cycles during sleep periods and all activities performed during awake periods. In fact, a person's performance in physical and mental activities is often dependent upon the amount and quality of sleep obtained before the activity. Recovery from injuries and harsh workouts can also be affected by the rest or sleep obtained during the recovery process. Accordingly, it is important to monitor sleep or rest activity as well as active workouts.

A user's physiological response during transition periods before and after sleep can also be informative of the user's overall physiological condition. For example, a small increase in resting heart rate upon waking can be an indicator that a person is over-training for an endurance activity. Accordingly, physiological characteristic monitoring during transition periods between sleep and awake activity can be an important indicator of a person's physiological condition.

Often a person's activity over a period of several days, weeks, months or longer may be indicative of that person's performance in a specific activity or the likelihood of a physiological event, such as a heart attack. Long term trends in physiological data can be used to predict athletic performance, sleep quality, cardiac failure and other conditions. Accordingly, a system that monitors physiological data over a long period of time can be used to predict physiological events.

Embodiments of the present invention can be used to monitor physiological characteristics during sleep, during transition to and from sleep and during awake activities over a period of many days. When this physiological data is recorded over many days, comparisons can be made to identify trends in the physiological data. When physiological events occur, such as a personal best in a triathlon event or a heart murmur, the physiological data preceding the event can be analyzed to identify a pattern in the data that can then be used to predict the physiological event.

When a physiological trend leading up to an adverse event, such as a heart attack, is identified, an alert can be sent to a user or another party warning of the impending event. Likewise, when a user deviates from a physiological trend that leads up to a positive event, an alert can be sent to the user or another party warning the user to return to the positive regimen.

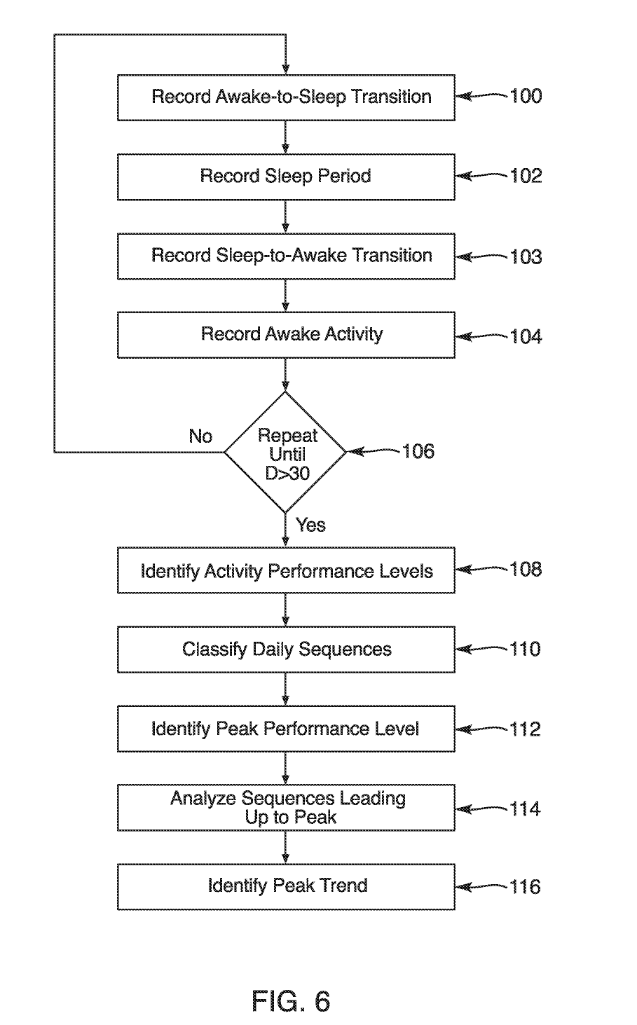

Some embodiments of the present invention may be described with reference to FIG. 6. In these embodiments, a record of physiological data for a user is recorded. This physiological data may be correlated with activities automatically, manually or may not be activity-correlated. This physiological data is accumulated over a time cycle divided into periods related to sleep conditions and/or physical activity. The exemplary embodiment of FIG. 6 begins with an awake-to-sleep transition period, however, the periodical physiological data can begin at any point in the record period.

In the exemplary embodiment of FIG. 6, the recording process begins at an awake-to-sleep (A/S) transition period 100, which may correspond to the period between laying down to sleep and achieving sleep. During this period, a user's physiological data is recorded 100. Once sleep is achieved, the user's physiological data continues to be recorded 102 during a sleep period. A sleep period may be further divided into light sleep, rapid-eye-movement (REM) sleep, lucid dreaming or other sleep categories based on brain activity or other parameters.

After a sleep period, a sleep-to-awake (S/A) transition period occurs and data for that transition is recorded 103 as well. In some embodiments, an S/A period may comprise increased brain activity, increased physical activity, a fixed period of time around such increased activity or other indicators of the termination of sleep and an associated time frame. An exemplary full periodical cycle is accomplished at the end of an awake activity period wherein a user's physiological data is recorded 104.

This data recording 100, 102, 103, 104 can be continued for multiple periodical cycles until some threshold number of cycles has transpired. When the threshold number is reached 106, recording may be terminated and the data may be analyzed. In this exemplary embodiment, activity performance levels may be identified 108. This process may comprise analysis of sleep cycles and identification of types of sleep, quality of sleep, restfulness, dream activity and other factors. This process may also comprise an analysis of awake activities, such as physical exercises and the physiological data recorded during those time frames may be tagged. In some embodiments, power output levels can be determined. In some embodiments, exercise heart rates can be compared to a theoretical maximum heart rate.

In some embodiments, daily or periodic sequences can be classified 110, so that periodic sequences can be compared. In some embodiments, daily sequences can be identified so that daily performance changes can be identified.

In some embodiments, a peak performance level can be identified 112. For example, a new personal best in a physical exercise, a peak volumetric blood flow rate, a high degree of rest during sleep or some other performance.

Once the peak performance has been identified 112, an analysis of the physiological data sequences leading up to that performance event may be analyzed 114 to determine the factors and changes in the physiological condition that led to the peak performance level. This analysis may comprise a statistical analysis of physiological data over many data sequences. This analysis may comprise the identification of trends that occur before a given peak performance event.

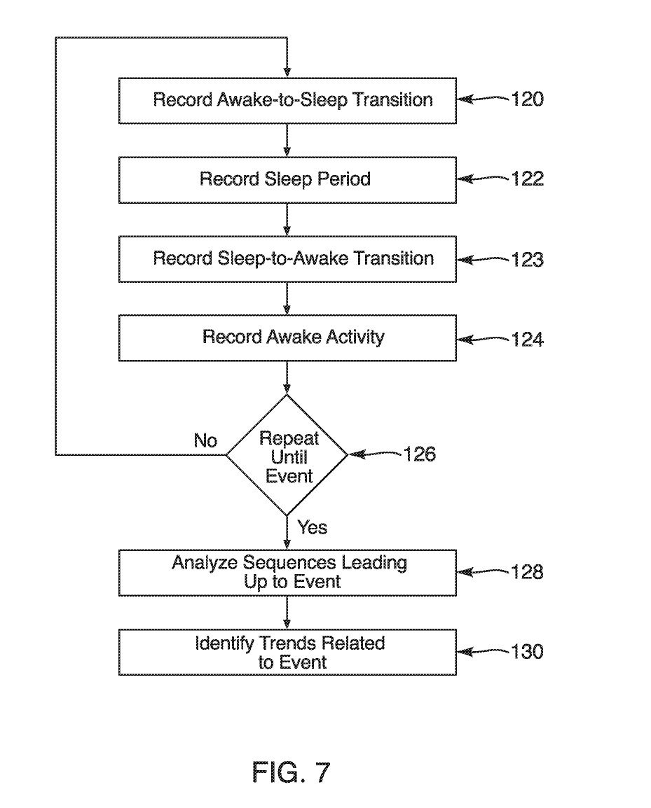

Some embodiments of the present invention may be described with reference to FIG. 7. In these embodiments, a record of physiological data for a user is recorded. This physiological data may be correlated with activities automatically, manually or may not be activity-correlated. This physiological data is accumulated over a time cycle divided into periods related to sleep conditions and/or physical activity. The exemplary embodiment of FIG. 7 begins with an awake-to-sleep transition period, however, the periodical physiological data can begin at any point in the record cycle.

In the exemplary embodiment of FIG. 7, the recording process comprises an awake-to-sleep (A/S) transition period 120. During this period, a user's physiological data is recorded 120. Once sleep is achieved, the user's physiological data continues to be recorded 122 during a sleep period.

After a sleep period, a sleep-to-awake (S/A) transition period occurs and data for that transition is recorded 123 as well. A full periodical cycle is accomplished at the end of an awake activity period wherein a user's physiological data is recorded 124.

This data recording 120, 122, 123, 124 can be continued indefinitely for multiple periodical cycles until some event occurs. When a designated event occurs 126, recording may be terminated and the data may be analyzed. In this exemplary embodiment, physiological data sequences leading up to the event may be analyzed 128. This process may comprise analysis of sleep cycles and identification of types of sleep, quality of sleep, restfulness, dream activity and other factors. This process may also comprise an analysis of awake activities, such as physical exercises and the physiological data recorded during those time frames may be tagged.

Analysis of the physiological data sequences leading up to the event may be performed 114 to determine the factors and changes in the physiological condition that led to the event. This analysis may comprise a statistical analysis of physiological data over many data sequences. This analysis may comprise the identification of trends 130 that occur before a given event.

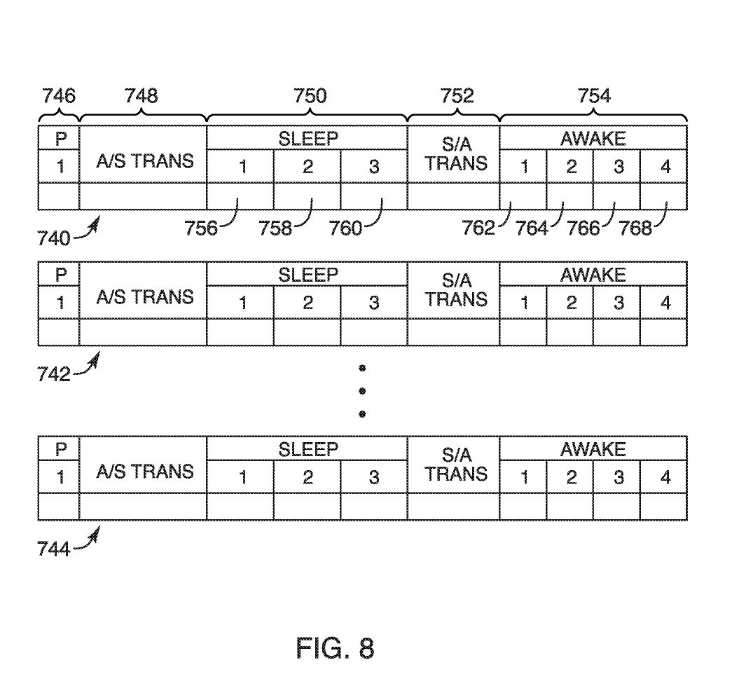

Some embodiments of the present invention may be described with reference to FIG. 8. These exemplary embodiments comprise a data record comprising periodical sequence records 140, 142, 144. Each periodical sequence record (PSR) 140, 142, 144 may comprise a sequence indicator 146 to identify the temporal position of the record in the series of sequences. A PSR 140 may also comprise an awake-to-sleep (A/S) transition record, which may comprise physiological characteristic values demonstrated during a period before sleep occurs. A PSR may also comprise a sleep record 150, which may comprise record values for various types of sleep 156, 158, 160 including, but not limited to, light sleep, REM sleep, dreaming, waking sleep and others.

An exemplary PSR 140 may further comprise a sleep-to-awake transition period record 152, where physiological data related to a period between sleep and an awake state is recorded. This record may comprise heart rate or brain activity data while the user is sleeping, immediately after waking and after a short period of being awake. A PSR 140 may further comprise an awake period record 154, which may be divided into multiple sub-periods 162, 164, 166, 168 based on the level of physical activity, heart rate, timing or other parameters.

Multiple PSRs 140, 142, 144 may be recorded continuously for each cycle or sequence of a periodic cycle or for intermittent sequences. Once the PSRs have been obtained, they may be analyzed to identify trends in the data.

Some embodiments of the present invention relate to analysis and comparison of recorded physiological data. These embodiments may comprise a multiplicity of historical data records or a database of records for a user and/or other individuals.

In some embodiments, these data records may comprise a multiplicity of records describing the physiological state of third-party individuals leading up to an event. These events may be positive or negative events, such as athletic success, coronary fitness, mental achievement or cardiac arrest, organ failure, disease and psychological depression. In some embodiments, these records may comprise sleep data, sleep transition data and awake activity data for a period of time before the event. In some embodiments, this data may be processed to find statistical or other trends that may indicate the imminent nature of the event. In some embodiments, these trends may be described in the form of an equation, a range of values or some other mathematical representation. In some embodiments, these trends may be represented as a histogram. Trends or trend data may be stored on a storage device, such as on a hard drive or in memory on mobile device 76, off-body computing device 78, cloud storage 88 or at some other storage device connected to mobile device 76 and/or wearable components 72, 74.

In some embodiments, demographic characteristics of the third-party data contributors may be used to match that data to a user. For example, third-party age, race, geographical location, gender, eating habits, weight, fitness level and other characteristics may be used to match third-party data trends to a user's personal data.

In some embodiments of the present invention, a user's current cumulative physiological data record can be compared to these trends to determine whether an event is likely to occur to the user. These comparisons may comprise comparisons of sleep data, sleep transition data, awake activity data and many physiological factors. These comparisons may also comprise physiological data correlated with an activity and further correlated with a trend. If a comparison does indicate that a positive event is likely to occur, a message may be sent to the user via mobile device 76 and/or wearable component 72 or 74. If a comparison indicates that a negative event is probable, a warning message may be sent to the user in a similar manner to warn the user of the imminent threat.

In some embodiments of the present invention, user physiological data, such as PSRs may be accumulated over a period of time. This user-specific data may then be analyzed with respect to events that have occurred in the life of the user to determine user-specific trends. This trend data may then be compared to current PSRs or other current physiological data to predict the probability of reoccurrence of those events. If the probability of reoccurrence is above a threshold level, a message may be sent to the user, as described above, to warn the user of a negative impending event or to encourage the user to continue activity leading to a positive event.

The present invention may be embodied in other specific forms without departing from its spirit or essential characteristics. The described embodiments are to be considered in all respects only as illustrative and not restrictive. The scope of the invention is, therefore, indicated by the appended claims, rather than by the foregoing description. All changes which come within the meaning and range of equivalency of the claims are to be embraced within their scope.

What is claimed and desired to be secured by Letters Patent is:

1. An apparatus for physiological characteristic monitoring during a sequence of sleep and activity, said apparatus comprising:
a wearable component comprising,
a physiological sensor for measuring a physiological characteristic of a wearer,
an accelerometer for measuring wearer activity, and
a wearable component wireless transceiver;
a mobile device comprising,
a processor and a memory, and
a mobile device wireless transceiver;
wherein said physiological sensor makes a plurality of physiological characteristic measurements;
wherein said wearable component transmits said measurements to said mobile device using said wireless transceivers;
wherein said mobile device records said plurality of measurements;
wherein said mobile device analyzes said plurality of measurements to identify periods of sleep, periods of activity and transition periods between sleep and activity for a plurality of cycles to create physiological sequence records and to identify physiological events; and
wherein said mobile device analyzes said physiological sequence records to identify trends leading up to said events.

2. The apparatus of claim 1 wherein said mobile device further uses said trends and said events to determine the probability of reoccurrence of said events.

3. An apparatus for physiological characteristic monitoring, said apparatus comprising:
a wearable component comprising,
a physiological sensor for measuring a physiological characteristic of a wearer,
an accelerometer for measuring wearer activity, and
a wearable component wireless transceiver;
a mobile device comprising,
a processor and a memory,
a display, and
a mobile device wireless transceiver; and
a database comprising,
a record of historical physiological characteristic data correlated with events;
wherein said physiological sensor makes a plurality of physiological characteristic measurements;
wherein said wearable component transmits said measurements to said mobile device using said wireless transceivers;
wherein said mobile device records said plurality of measurements; and
wherein said mobile device compares said plurality of measurements to said record of historical physiological characteristic data to determine the probability of reoccurrence of one or more of said events.

4. The apparatus of claim 3 wherein said mobile device further alerts a user of an impending event when said probability of reoccurrence is high.

5. The apparatus of claim 3 wherein said database is located at an off-body computing device.

6. An apparatus for physiological characteristic monitoring, said apparatus comprising:
a wearable component comprising,
a physiological sensor for measuring a physiological characteristic of a wearer,
an accelerometer for measuring wearer activity, and
a wearable component wireless transceiver;
a mobile device comprising,
a processor and a memory,
a display, and
a mobile device wireless transceiver; and
an off-body computing device comprising,
a processor and memory,
a storage device,
an off-body device wireless transceiver, and
a record of historical physiological characteristic data correlated with events;
wherein said physiological sensor makes a plurality of physiological characteristic measurements;
wherein said wearable component transmits said measurements to said mobile device using said wireless transceivers;
wherein said mobile device transmits said measurements to said off-body computing device;
wherein said off-body computing device records said plurality of measurements; and
wherein said off-body computing device compares said plurality of measurements to said record of historical physiological characteristic data to determine the probability of reoccurrence of one or more of said events.

7. A method for physiological characteristic monitoring, said method comprising:
measuring a physiological characteristic of a wearer with a wearable component comprising,
a physiological sensor for measuring said physiological characteristic of a wearer,
an accelerometer for measuring wearer activity, and
a wearable component wireless transceiver;
measuring wearer activity data with said accelerometer;
sending said physiological characteristic and said wearer activity data to a mobile device, said mobile device comprising,
a processor and a memory,
a display, and
a mobile device wireless transceiver;
sending said physiological characteristic and said wearer activity data from said mobile device to an off-body computing device comprising,
a processor and memory,
a storage device,
an off-body device wireless transceiver, and
a record of historical physiological characteristic data correlated with events;
recording said physiological characteristic and said wearer activity data at said off-body computing device; and
comparing said physiological characteristic and said wearer activity data to said record of historical physiological characteristic data correlated with events to determine the probability of reoccurrence of one or more of said events based on said physiological characteristic and said wearer activity data.
